(19) United States
(12) Patent Application Publication
(10) Pub. No.: US 2020/0400667 A1
(43) Pub. Date: Dec. 24, 2020

(54) METHOD FOR THE QUANTIFICATION OF MEASLES AND RUBELLA TARGETS

(71) Applicant: InDevR, Inc., Boulder, CO (US)

(72) Inventors: Kathy L. ROWLEN, Boulder, CO (US); Rose BYRNE-NASH, Boulder, CO (US); Jacob GILLIS, Boulder, CO (US)

(73) Assignee: InDevR, Inc., Boulder, CO (US)

(21) Appl. No.: 16/906,373

(22) Filed: Jun. 19, 2020

Related U.S. Application Data
(60) Provisional application No. 62/864,847, filed on Jun. 21, 2019.

(51) Int. Cl. G01N 33/569; G01N 33/58; G01N 1/40

(52) U.S. Cl. CPC G01N 33/56983; G01N 33/582; G01N 2458/00; G01N 2333/19; G01N 2333/12; G01N 1/4077

(57) ABSTRACT

Provided herein is a method for multiplexed detection of a plurality of targets, including targets associated with a measles (M) virus and a rubella (R) virus, including a M vaccine, a R vaccine, or a MR vaccine. A plurality of capture agents specific to a measles target and a rubella target are provided on a substrate, wherein the capture agents specifically bind to the measles target and the rubella target. Contacting the plurality of capture agents with a sample forms a capture agent-target complex which can be detected by a corresponding spatial pattern of capture agent-target complex.

### CROSS-REFERENCE TO RELATED APPLICATIONS

This application claims the benefit of priority to U.S. Provisional Patent Application No. 62/864,847 filed on Jun. 21, 2019, which is specifically incorporated by reference to the extent not inconsistent herewith.

### BACKGROUND

Measles virus is in the Paramyxoviridae family, and is an enveloped, negative strand RNA virus with a non-segmented genome. Measles virus has two surface proteins, hemagglutinin (HA) and fusion (F), with HA responsible for attachment to host cell receptors, and F responsible for fusion with plasma membrane and subsequent ribonucleocapsid release. The measles nucleoprotein (NP) encapsidates the genome and is also thought to inhibit the immune response when released into the blood following lysis of measles-infected cells. Measles virus is one of the most contagious of the vaccine-preventable diseases and is transmitted via the respiratory route. While often self-limiting, measles virus infection can cause complications and can result in an immune suppression that can last up to 2 years post-infection and cause susceptibility to other infections and increased risk of mortality. In industrialized countries, case fatality for measles is ˜0.1%, but in developing countries, the case fatality rate approaches 2-8%.

Rubella virus is in the Togaviridae family and is an enveloped positive-strand RNA virus with a non-segmented genome. The rubella virus has two protein spikes on the virion surface, E1 and E2. Trimers of glycosylated E1-E2 mediate attachment to host cell receptors, and a capsid (C) nucleoprotein surrounds the genome with icosahedral symmetry and is key for successful replication in host cells. Rubella viral infection is generally mild clinically and less infectious than measles, but infections during pregnancy can result in congenital rubella syndrome which causes severe birth defects. In addition, sub-clinical rubella infections can still be contagious to the non-vaccinated and immunologically naïve. It is estimated that ˜100,000 cases of congenital rubella syndrome occur every year.

While measles and rubella are largely prevented with effective vaccines in the industrialized world, these diseases remain poorly controlled in the developing world due to lack of proper immunization programs, recently organized programs, lack of effective surveillance, lack of appropriate resources and logistical support, among other reasons. Due to the highly contagious nature of measles, 95% vaccine coverage is needed for effective control. The global Measles and Rubella Strategic Plan (2012-2020) includes eventual goals for the eradication of both diseases, with reasonable progress made towards these goals. While mumps virus is often associated with measles and rubella due to the combination in the common trivalent measles-mumps-rubella (MMR) vaccine, mumps has not been targeted for global eradication, and many nations do not consider mumps to be a priority for vaccination.

Live, attenuated bivalent measles-rubella (MR) vaccine is targeted for the developing world, and an Indian-manufactured vaccine is used extensively, with 150 million doses distributed, and goals to increase distribution further to increase vaccination rates towards disease eradication. A combined live attenuated trivalent MMR vaccine was first introduced by Merck in 1971, with a two-dose regimen commonly employed in many industrialized nations (one dose at <2 years of age, and one generally given at age 3+, depending on the specific region). Currently, the National Institute of Biologicals (India) outline that identity and potency testing of measles, rubella, and bivalent measles-rubella (MR) vaccine are cell line-based, typically performed by tissue culture infectious dose, or TCID50. In addition, vaccine stability tests of vaccine exposed to 37° C. are also performed via TCID50. Not only is TCID50 used in testing of the final vaccine formulation but is also used at a variety of steps in the production process including after harvest, concentration, filtration, blending, and lyophilization. Three replicates of TCID50 are performed for each virus in both identification/potency and stability assays. At times, lot rejections occur after harvest and after stability testing, with the imprecision in TCID50 being a contributing factor to lot rejections.

As stated, the current standard method for virus quantification in MR vaccines is tissue culture infectious dose, or TCID50. The TCID50 assay is an endpoint dilution assay utilized to determine infectious titer of a virus that causes cytopathic effect (CPE) in tissue culture, with TCID50 defined as the amount of virus producing CPE in 50% of infected tissue culture cells. The assay generally involves plating replicate wells with a known number of cells and adding serial dilutions of the virus. After incubation, the wells are manually observed to determine the percentage of CPE, and these results used to calculate the TCID50 value. TCID50 is a slow, labor intensive, biological assay to quantify viruses, requiring ˜10 days to complete for measles and rubella. In addition, the TCID50 assay is singleplex, requiring different assays to be conducted for both measles and rubella, as well as requiring highly trained personnel, good aseptic technique and specialized facilities. In addition, the method employs subjective, manual methods for readout that lead to significant imprecision.

Desirable features for antigen quantification for MR-containing vaccines include high-throughput and multiplexing capability to enable separate quantification of each of the components of a multivalent vaccine in a single test, rapid target antigen characterization during various steps of upstream processing and purification, and capability for stability indication (the extent of degradation upon stress such as thermal) of each component of a multivalent vaccine.

Given the issues with current biological cell line-based identity, potency, and stability assays such as TCID50, there is a significant need for improvements to the technology for quantification of targets (e.g., antigen, protein, virus, etc.) in vaccines. In particular, there is a significant need for a multiplexed quantification method that is able to identify, differentiate between, quantify components of multivalent vaccines, and measure vaccine degradation for stability testing. An alternate target antigen or protein quantification method should also be more reliable and simpler to perform than the current method, with improved precision.

### SUMMARY

Provided herein are systems and methods for target (e.g., antigen) identity determination and quantification. The provided systems and methods may be multiplexed to determine target (e.g., antigen) content of a multivalent vaccine and for determining simultaneously multiple targets for a single vaccine. The provided systems and methods may also utilize multiple capture agents specific to different targets (e.g., antigens and/or proteins) from a single virus to enhance vaccine sample characterization. Also provided herein are systems and methods for cleanup and concentration of vaccine antigen(s) prior to identity testing or quantification, thereby improving quantification in a final vaccine. Reduced testing time offered by the systems and methods herein may reduce costs and may allow producers to test vaccine samples during various stages of the production process, allowing for recognition of problematic batches earlier and optimization of process steps due to greater insight into process results.

Accordingly, several advantages of one or more aspects of the technology over conventional TCID50 methods include, but are not limited to, the following: to rapidly quantify proteins or vaccine antigens to enhance vaccine development, characterization, or release, to identify one or more antigens in a multivalent vaccine using a single test, to quantify one or more antigens in a multivalent vaccine using a single test, to measure stability of a vaccine component or multiple vaccine components, to eliminate the use of TCID50 in the identity, potency, or stability testing in a vaccine, to provide a low-cost, simple testing method that can be performed by a user with minimal technical expertise, to increase precision of target (e.g., antigen) quantification, and to simplify vaccine target (e.g., antigen) quantification and reduce costs. Other advantages of one or more aspects will be apparent from a consideration of the drawings and ensuing description.

Any of the systems and methods provided herein may be array-based, allowing for multiplexing capability, and providing for a reduction in testing time due to ability to simultaneously assay for multiple targets. The array provided herein is compatible with a range of configurations, including an array on a flat substrate, a bead-based array, or any other type of array commonly known in the art.

The method may be for identification of targets in a vaccine sample, the targets comprised of, or corresponding to, any one or more of antigens, proteins, virions, viruses, or virus-like particles such as by: 1) providing a substrate with one or more capture agents, 2) contacting the substrate with a vaccine sample to form a bound complex between the capture agent and a target, 3) washing away unbound material in the vaccine sample, 4) labeling the bound complex with one or more label agents so as to produce a detectable signal, 5) and detecting the presence of vaccine component in a vaccine sample

The method may include quantification of targets in a vaccine sample, the targets may correspond to components useful in vaccine characterization, evaluation and assessment, such as comprising one or more of antigens, proteins, virions, viruses, or virus-like particles. The methods and systems provided herein may be particularly useful for assessment, at various points, along a vaccine manufacturing process. For example, a measles or a rubella vaccine production process may be assessed at various steps along the process, including relatively upstream when before the vaccines may have been combined. This reflects the reliability, sensitivity, and relatively rapid turn-around time of the instant assays compared to conventional TCID50 assays. This can be invaluable for identifying potential problems upstream, thereby saving significant time and costs if the test was more downstream in the manufacturing process.

Provided is a method for multiplexed detection of a plurality of targets associated with a measles virus and a rubella virus, the method comprising the steps of: providing a plurality of capture agents specific to a measles target and a rubella target, wherein the capture agents specifically bind to the measles target and the rubella target; contacting the plurality of capture agents with a sample, wherein measles target and rubella target in the sample form a capture agent-target complex; and detecting a spatial pattern of capture agent-target complex.

The capture agents can be any of a wide range of types, so long as the capture agent is capable of specifically binding to a target. Examples include antibodies, such as monoclonal antibodies. “Specific binding” refers to the capture agent that binds to the desired target, with minimal binding to other targets or components in a vaccine, including minimal non-specific binding. Specific binding can be characterized as the ability of the target to generate detectable signals on capture agents expected to bind the target while simultaneously generating signal of less than 3 times background on capture agents not expected to bind the target. In particular, the target is run at a concentration at or above the middle of the linear dynamic range, that is, at a concentration resulting in 50% of the saturating signal obtained at concentrations exceeding the linear dynamic range, to provide a detectable amount of bound complexes and an appropriate label agent known to bind the target is used so that the detected signal is robust and not at or near a detection limit (e.g., is above at least about 3 to 10 times background).

“Label agent” refers to a label that is useful to detect a bound (capture agent)-(target) complex. Any of a range of label agents may be used, including radiolabels, optical labels or the like. A preferred label agent is a fluorescent label that can bind to a target, wherein the target is, in turn, bound to the capture agent.

Preferably, the methods further comprise quantification of a target in the sample, such as an antigen or virus in a vaccine. Quantification may be achieved by using any of the methods and systems described in US Pub. No. 2017/0199192 titled “Universal Capture Array for Multiplexed Subtype-Specific Quantification and Stability Determination of Influenza Proteins” (and corresponding PCT Pub. WO 2015/187158); U.S. Pat. No. 10,261,081; which are specifically incorporated by reference herein for the array layouts, standardized dilution applications, and resultant calibration curves.

In one embodiment, one or more calibration curves are constructed using one or more arrays 102 and one or more standardized target dilutions at known concentrations. One or more standardized target dilutions at known concentrations are contacted with one or more arrays 102, and a signal that correlates with the amount of bound complex between capture agent(s) and target(s) is generated and quantified using appropriate methods to form one or more calibration curves that yield a relationship between quantity of fluorescence signal of capture agent and concentration of target. In one embodiment, 7 different concentrations of a serially-diluted standardized target may be contacted with 7 different arrays 102 to form bound complexes that are then labeled with an appropriate label agent, and the fluorescent signal may be quantified using a microarray imaging system to form calibration curves that yield a relationship between quantity of fluorescence signal on the capture agent(s) and initial absolute concentration of the target(s) in the vaccine sample.

Representative embodiments of the invention are provided by the claims appended herein, which are specifically incorporated by reference into this specification.

### BRIEF DESCRIPTION OF THE DRAWINGS

Non-limiting and non-exhaustive embodiments of the technology of the present application, including the preferred embodiment, are described with reference to the following figures, wherein like reference numerals refer to like parts throughout the various views unless otherwise specified.

FIG. 1 shows one embodiment of a substrate including replicate arrays consistent with the present disclosure.

FIG. 2 shows a schematic illustration of a capture agent bound to a substrate that specifically binds the relevant target. In this illustration the capture agent is antigen-specific and the target is a viral antigen. A label agent may be used to bind to the target (e.g., viral antigen), such as a fluorophore-conjugated label agent.

FIG. 3 shows graphs of the reactivity of capture agents (nine distinct anti-measles antibody) to measles target according to one embodiment of the present disclosure.

FIG. 4 shows an image of the reactivity of rubella target captured by anti-rubella antibody capture agents and lack of cross-reactivity of rubella target on anti-measles antibody capture agents (capture agent layout summarized in FIG. 1) according to one embodiment of the present disclosure.

FIG. 5 shows graphs of response curve linearity and linear dynamic range of rubella target captured by anti-rubella antibody capture agents that target E1, E2, and C proteins of rubella virus according to one embodiment of the present disclosure.

FIG. 6 shows an image of the reactivity of measles target captured by anti-measles antibody capture agents and lack of cross-reactivity of measles target on anti-rubella antibody capture agents (capture agent layout summarized in FIG. 1) according to one embodiment of the present disclosure.

FIG. 7 shows a graph of the response curve linearity for measles target captured by an anti-measles antibody capture agent according to one embodiment of the present disclosure.

FIG. 8 shows a graph of a correlation for rubella target quantification between the array-based method consistent with the methods and systems of the present disclosure and a real-time RT-PCR assay analysis.

FIG. 9 shows a graph of a correlation for rubella target quantification between the array-based method consistent with the methods and systems of the present disclosure and a TCID50 assay analysis.

FIG. 10 shows a graph of the comparative quantification of rubella target between the array-based method consistent with the methods and systems of the present disclosure, a real-time RT-PCR assay analysis, and a TCID50 assay analysis.

FIG. 11 shows a graph of an analysis of the precision for rubella target quantification of an array-based method consistent with the present disclosure over 3 individual days at two different sample dilutions according to one embodiment of the present disclosure.

FIG. 12 shows a graph of a correlation for measles target quantification between the array-based method consistent with the methods and systems of the present disclosure and a real-time RT-PCR assay analysis.

FIG. 13 shows a graph of a correlation for measles target quantification between the array-based method consistent with the methods and systems of the present disclosure and a TCID50 assay analysis.

FIG. 14 shows a graph of the comparative quantification of measles target between the array-based method consistent with the methods and systems of the present disclosure, a real-time RT-PCR assay analysis, and a TCID50 assay analysis.

FIG. 15 shows a graph comparing the response curves for rubella target in a monovalent sample and a bivalent sample comprised of rubella target and measles target according to one embodiment of the present disclosure.

FIG. 16 shows a graph comparing the response curves for measles target after a pre-processing step in a monovalent sample and a bivalent sample comprised of rubella target and measles target according to one embodiment of the present disclosure.

FIG. 17 shows a graph comparing the % expected (% recovery) of measles target after exposure to both a spin-based pre-processing (pre-concentration and cleanup) method and a magnetic-based method according to one embodiment of the present disclosure.

FIG. 18 shows a graph of the stability indicating behavior of anti-rubella antibody capture agents for rubella target that has been exposed to a heat degradation protocol according to one embodiment of the present disclosure.

FIG. 19 shows a graph of the stability indicating behavior of anti-measles antibody capture agents for measles target that has been exposed to a heat degradation protocol according to one embodiment of the present disclosure.

FIG. 20 is a plot of measured titer (IFU/mL) as a function of expected titer (IFU/mL) for a without preprocessing of Example 8 assay (circles) and with preprocessing of Example 8 (diamonds”) for measles.

FIG. 21 is a plot of measured titer (IFU/mL) as a function of expected titer (IFU/mL) for a without preprocessing of Example 8 assay (circles) and with preprocessing of Example 8 (diamonds) for rubella.

### DETAILED DESCRIPTION

The technology of the present application will now be described more fully below with reference to the accompanying figures, which form a part hereof and show, by way of illustration, specific exemplary embodiments. These embodiments are disclosed in sufficient detail to enable those skilled in the art to practice the technology of the present application. However, embodiments disclosed herein may be implemented in many different forms and should not be construed as being limited to the embodiments set forth herein. The following detailed description is therefore, not to be taken in a limiting sense. Moreover, the technology of the present application will be described with relation to exemplary embodiments. The word “exemplary” is used herein to mean “serving as an example, instance, or illustration.” Any embodiment described herein as exemplary is not necessarily to be construed as preferred or advantageous over other embodiments. Additionally, unless specifically identified otherwise, all embodiments described herein should be considered exemplary.

For the purposes of the current invention, the term target refers to an antigen, protein, virus, virion, virus-like particle, or other vaccine component that is intended for capture by the capture agents on the array of the current invention.

In one embodiment the substrate with replicate arrays is manufactured by printing methods conventionally used for manufacturing high- or low-density flat substrate microarrays, for example non-contact microarray printing such as inkjet or piezoelectric printing. Other manufacturing methods are also possible including, but not limited to chemical binding of capture agents to microsphere (“bead”) surfaces in solution or in air, or other array and manufacturing methodologies known in the art.

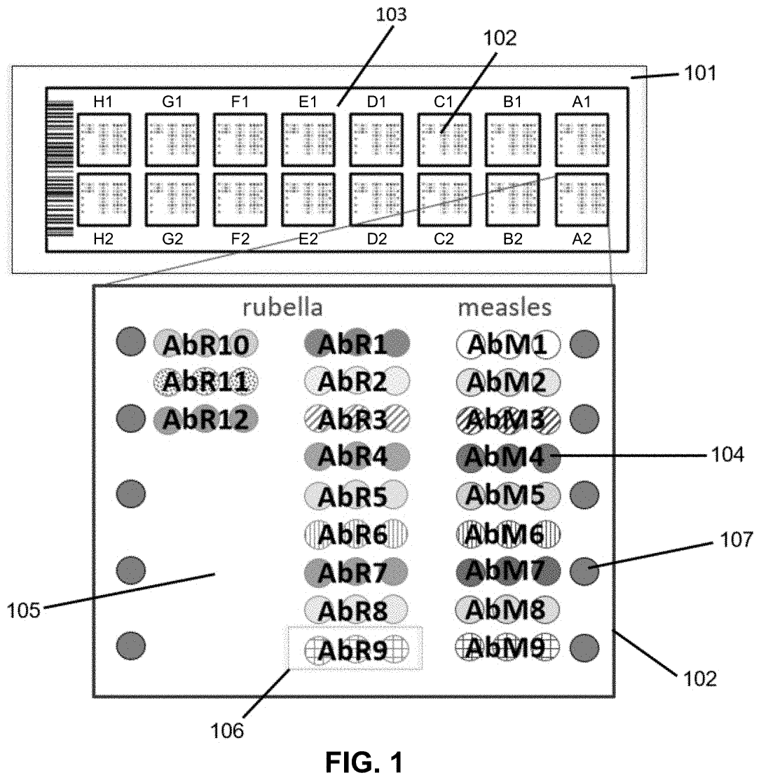

In one embodiment, and as illustrated in FIG. 1 (left), multiple replicates of the array 102 may be printed on a substrate 101. The substrate 101 may be approximately 25 mm×75 mm in lateral dimensions and 0.5 mm in thickness. However, in other embodiments substrate 101 may be a different size, for example 25 mm×25 mm in lateral dimensions and 0.5 mm in thickness, or 1 cm by 1 cm in lateral dimensions with thickness 1 mm. In another embodiment, the array 102 may be of a non-rectangular shape, and be printed on a non-glass substrate such as a flexible polymer or other flexible substrate that may be produced via roll-to-roll manufacturing. In another embodiment, the array 102 may be printed in the well of a 24-, 48-, 96-, or 384-well plate.

In one embodiment, and as illustrated in the top panel of FIG. 1, there may be 16 replicate arrays 102 printed on a single substrate 101. However, in other embodiments, there may be alternate numbers and configurations of replicate arrays 102 on the substrate 101. The bottom panel of FIG. 1 is a close-up view of a single replicate array 102.

In one embodiment, the substrate 101 may be additionally outfitted with a gasket 103. In some embodiments, the gasket 103 provides a visual indicator of where the replicate arrays are located on the substrate. In some embodiments, the gasket 103 acts as a reaction vessel that confines a predetermined amount of a vaccine sample or other liquid material to a single array 102.

In one embodiment, and as illustrated in FIG. 1 (bottom), an array 102 is ˜3×4.3 mm square, with individual spots 104 ranging from 50-400 micrometers in diameter, patterned in a regularly-spaced rectangular array. In another embodiment, some individual areas on the array 105 may be printed with a buffer (“blank” spot) or may be empty and not have a spot printed in the nominal spot location. However, one who is skilled in the art will recognize that many alternative sizes, patterns, shapes and spacings of possible, both regularly- and irregularly-spaced and rectangular and non-rectangular.

In one embodiment, one or more spots 104 containing multiple copies of a single capture agent 106 may be printed on the array 102, with another spot or other spots containing a different capture agent, as denoted by the AbM1, AbM2 . . . AbM9 and AbR1-AbR9 notation for a measles and rubella capture agents, respectively in FIG. 1. In one embodiment, there may be 3 replicate spots of each capture agent, as illustrated by capture agent 106 in FIG. 1 (each containing replicates as indicated by the three spots 104). In other embodiments, there may be up to 12 replicate spots of each capture agent (e.g., there would be 12 spots associated with each of the capture agents 106 AbR1 . . . AbM9, instead of the three spots illustrated in FIG. 1). In an embodiment, all replicates of a single capture agent may be referred to as a sub-array (e.g., corresponding to the “rectangle” associated with 106). One skilled in the art will recognize that alternative numbers of replicate spots for each capture agent are also possible. If additional multiplexing is desired, such as to detect another relevant component of a vaccine (e.g., measles, etc.) or an unwanted contaminant, additional sub-arrays can be provided where the capture agent specifically binds to the additional component (e.g., measles, contaminant biological material, etc.). Of course, the methods provided herein are compatible with any of a large number of arrangements of sub-array layouts and capture agent types, including antibody capture agents, so long as during read-out, the underlying capture agent associated with the position of each subarray 106 is known.

Exemplary measles capture agents include, but are not limited to, one or more of Novus Biologicals (Centennial, Colo.) anti-measles antibody NB110-3507, LSBio® (Seattle, Wash.) anti-measles antibody LS-C75585, LSBio® anti-measles antibody LS-C744343, LSBio® anti-measles antibody LS-C744342, LSBio® anti-measles antibody LS-C744341, LSBio® anti-measles antibody LS-C744340, LSBio® anti-measles antibody LS-C683114, EastCoast Bio (North Berwick, Me.) anti-measles antibody HM402, anti-measles antibody LS-C683112, EastCoast Bio anti-measles antibody HM403, Fitzgerald (Acton, Mass.) anti-measles antibody 10-001605F, and/or ViroStat (Westbrook, Me.) anti-measles antibody 6012. In an embodiment, two or more, three or more, four or more, five or more, six or more, or between two and seven measle capture agents are used, including two, three or four measle capture agents.

Exemplary rubella capture agents include, but are not limited to, GeneTex® (Irvine, Calif.) anti-rubella antibody GTX39194, GeneTex anti-rubella antibody GTX10013, ViroStat anti-rubella antibody 1712, ViroStat anti-rubella antibody 1714, ViroStat anti-rubella antibody 1715, ViroStat anti-rubella antibody 1717, Antibodies-Online (Limerick, Pa.) anti-rubella antibody ABIN809899, Antibodies-Online anti-rubella antibody ABIN1684474, Antibodies-Online anti-rubella antibody ABIN1684475, Millipore Sigma (Burlington, Mass.) anti-rubella antibody MAB925, HyTest (Turku, Finland) anti-rubella antibody 3R23-Ru6, HyTest anti-rubella antibody 3R23-1011.

In one embodiment, capture agents 106 are selected from the group consisting of one or more of AbM1 (Novus Biologicals anti-measles antibody NB110-3507), AbM2 (LSBio anti-measles antibody LS-075585), AbM3 (LSBio anti-measles antibody LS-0744343), AbM4 (LSBio anti-measles antibody LS-0744342, AbM5 (LSBio anti-measles antibody LS-0744341), AbM6 (LSBio anti-measles antibody LS-0744340), AbM7 (LSBio anti-measles antibody LS-0683114 or EastCoast Bio anti-measles antibody HM402), AbM8 (anti-measles antibody LS-0683112 or EastCoast Bio anti-measles antibody HM403), AbM9 (Fitzgerald anti-measles antibody 10-001605F), and selected from the group consisting of one or more of AbR1 (GeneTex anti-rubella antibody GTX39194), AbR2 (GeneTex anti-rubella antibody GTX10013), AbR3 (ViroStat anti-rubella antibody 1712), AbR4 (ViroStat anti-rubella antibody 1714), AbR5 (ViroStat anti-rubella antibody 1715), AbR6 (ViroStat anti-rubella antibody 1717), AbR7 (Antibodies-Online anti-rubella antibody ABIN809899), AbR8 (Antibodies-Online anti-rubella antibody ABIN1684474), AbR9 (Antibodies-Online anti-rubella antibody ABIN1684475), AbR10 (Millipore Sigma anti-rubella antibody MAB925), AbR11 (HyTest anti-rubella antibody 3R23-Ru6), and AbR12 (HyTest anti-rubella antibody 3R23-1011).

In another embodiment, a smaller set of measles capture agents are selected for use on the array based on their characteristics. It is advantageous to select the least number of unique capture agents without unduly impacting sensitivity and reliability to decrease manufacture complexity and related costs. Preferred capture agents include those that result in the best sensitivity (also described as the ability to detect the lowest concentration of measles antigen possible). Such sensitivity may be described in terms of infectious units per milliliter (IFU/mL), with a limit of detection <1000 IFU/mL, and more ideally <500 IFU/mL, and even more ideally <50 IFU/mL. Alternatively, preferred capture agents include those that provide the highest specificity (also described as capture agents that have low binding affinity for non-target antigens or species, such as a measles binding agent that does not detect rubella antigen or any other non-measles species even when present at very high concentrations). Such specificity may be described in terms of a difference in signal generated for the target compared to non-targets typically found in the sample under test, such as a vaccine sample or a sample that is a step in a vaccine manufacturing process. For example, a difference in signal of 500× or 1000× or greater indicates good specificity. Alternatively, preferred capture agents include those that provide wide linear dynamic ranges (also described as the range of concentrations over which an antigen bound to the capture agent shows a linear change in signal as a function of concentration). Such linear dynamic ranges may be at least 100×, and more ideally at least 1000×. In this manner, capture agents are selected based on the capture agent sensitivity, specificity and/or linear dynamic range, and may depend on the application of interest where certain of the parameters are of higher importance than others.

In one embodiment, the measles capture agents on the array are selected from the group consisting of one or more of: EastCoast Bio anti-measles antibody HM403, EastCoast Bio anti-measles antibody HM402, NovusBio anti-measles antibody NB110-3507, and ViroStat anti-measles antibody 6012.

In another embodiment, the measles capture agents on the array include at least Novus Bio anti-measles antibody NB110-3507 and/or EastCoast Bio anti-measles antibody HM403.

In one embodiment, the rubella capture agents on the array include at least one or both of: Antibodies-online anti-rubella antibody ABIN809899 and Millipore Sigma anti-rubella antibody MAB925.

In another embodiment, the rubella capture agent on the array includes at least Antibodies-online anti-rubella antibody ABIN809899.

In another embodiment, capture agents on the array include at least Novus Bio anti-measles antibody NB110-3507 and/or EastCoast Bio anti-measles antibody HM403 and further includes Antibodies-online anti-rubella antibody ABIN809899 and/or Millipore Sigma anti-rubella antibody MAB925.

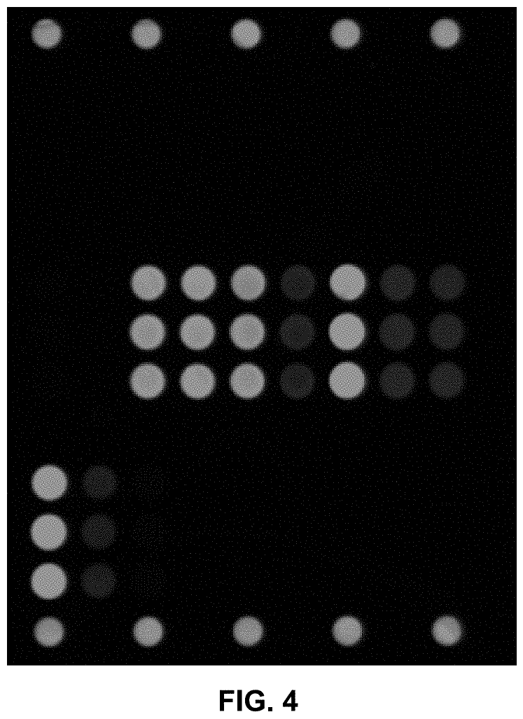

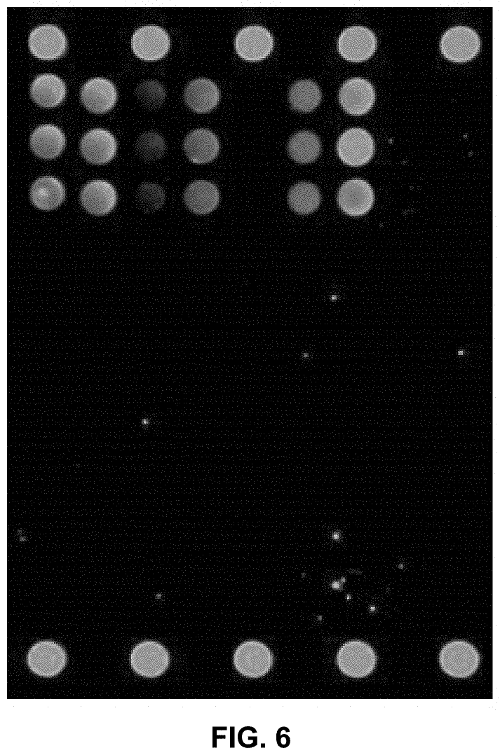

In one embodiment, the array 102 may include control spots 107 that may be used as fiducial markers to locate the array and aid in data analysis. For example, spots 107 can be identified in FIGS. 4 and 6.

In addition, arrays may be comprised of a plurality of capture agents bound to a plurality of microspheres (a single capture agent per microsphere) that are spatially distinct but free to move relative to one another.

In one embodiment, the capture agent is a monoclonal antibody. In one embodiment, the capture agent is a commercially-available monoclonal antibody. In another embodiment, the capture agent is a monoclonal antibody specifically developed and screened to bind the target set(s) of interest. In some embodiments, the capture agent spot includes one or more monoclonal antibodies configured to enable quantification of multiple targets from both measles and rubella viruses simultaneously.

In one embodiment, the monoclonal antibodies configured to enable quantification of multiple targets from measles virus are monoclonal antibodies targeting one or more of the following: nucleoprotein (NP), hemagglutinin (HA), or fusion protein (F). In one embodiment, the monoclonal antibodies configured to enable quantification of multiple targets from rubella virus are monoclonal antibodies targeting one or more of the following: E1 protein, E2 protein, or capsid protein (C).

In some embodiments, target quantification from measles virus does not require prior neutralization of rubella virus with antiserum.

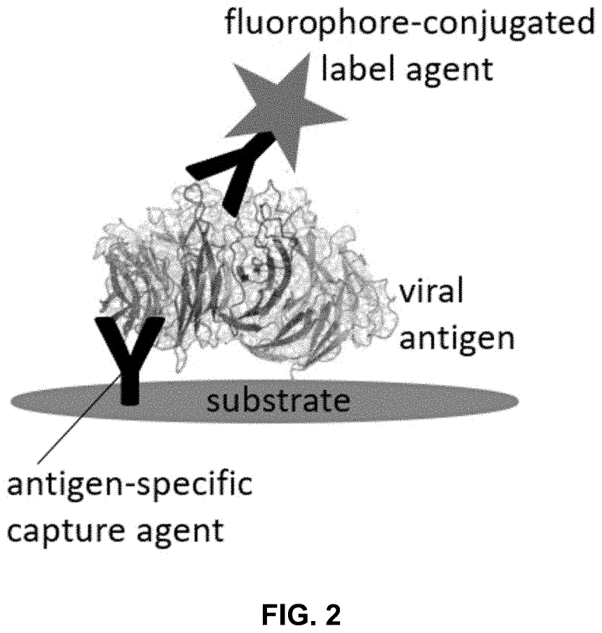

FIG. 2 illustrates the operation of one embodiment of the array shown in FIG. 1. In FIG. 2, a target-specific capture agent spot is printed on a substrate. Target-specific capture agent has affinity for a target in a vaccine sample, which binds to form a capture agent-target complex. In one embodiment, unbound target and other components of a vaccine sample are washed away. In another embodiment, a fluorophore-conjugated label agent is introduced to the array that has affinity for the target, which binds to the target. In another embodiment, excess or unbound fluorophore-conjugated label agent is washed away. A signal that correlates with the amount of bound complex at each spot is then generated and quantified. In one embodiment, a fluorescence-based sandwich-type immunoassay assay such as that illustrated in FIG. 2 is used to generate a fluorescence signal at each spot that correlates with the amount of bound complex at each spot. One skilled in the art will recognize that label agents other than those conjugated to a fluorophore are possible, such as but not limited to colorimetric label agents that undergo an enzymatic reaction producing a colored product, nanoparticle-based, or quantum dot-based label agents.

In one embodiment, the systems and methods herein may be used to identify the presence of the target, including a plurality of targets, including from a plurality of viral organisms. A fluorescence signal on the array for a specific capture agent or multiple capture agents after a vaccine sample has been applied may be utilized in this embodiment as an identity test for the presence of the desired target in the vaccine sample.

In one embodiment, the systems and methods described herein may be used to quantify a target. A calibration curve may be generated by applying dilutions of a standard target to replicate arrays on a substrate. Vaccine samples with unknown target concentration can also be applied to other replicate arrays on the same substrate or on a different substrate. The method of this embodiment then comprises labeling the bound target on all replicate arrays with an appropriate label agent, determining a relationship between the fluorescence signals generated from the replicate arrays to which dilutions of standard target were applied, and utilizing the fluorescence signals as a calibration curve with which to calculate the target concentrations in the vaccine samples with unknown target concentration. In another embodiment, the analysis of the fluorescence signals from the calibration curve and vaccine samples is automated with a software algorithm. See, e.g., US Pub. No. 2017/0199192, for quantification of influenza.

In one embodiment, the quantification of target can be used to determine concentration of one or more targets during vaccine process development or optimization. In another embodiment, the quantification of target can be used to determine potency of a vaccine.

In one embodiment, a pre-concentration or cleanup step may be conducted on the vaccine sample prior to quantification. In some embodiments, the pre-concentration or cleanup step may involve a centrifugation-based or magnetic bead-based protocol comprised of mixing the vaccine sample with an appropriate pre-concentration or cleanup matrix, allowing the target to bind to the matrix, centrifuging the mixture or applying a magnetic field to separate the matrix, removing the supernatant, resuspending the matrix with bound targets in a lysis mixture to lyse the target from the particles, centrifuging the matrix or applying a magnetic field to separate the matrix, and removing the supernatant containing the target. In some embodiments the supernatant containing the target is then applied to the array for quantification.

In one embodiment, the systems and methods of the current invention may be utilized to simultaneously quantify multiple targets. In some embodiments, the targets are from measles and rubella viruses. In some embodiments, the targets are from measles, mumps, and rubella viruses.

In one embodiment, the system and methods described herein may be used to determine the stability of a vaccine sample after a challenge is encountered, such as exposure to heat, pH changes, or other degradation protocols known to one skilled in the art. An aliquot of a vaccine sample may be used as a control, and another aliquot of a vaccine sample may be exposed to a degradation protocol such as exposure to 37 C for 7 days. After exposure to the degradation protocol, the control and degraded aliquots of vaccine sample may be analyzed alongside a calibration curve generated by applying dilutions of a standardized target to replicate arrays. The signals resulting from the dilutions of the standardized target may then be used to quantify the target in the control vaccine sample and degraded vaccine sample to measure stability. In some embodiments, the comparative quantification of target in a control and degraded vaccine sample can be used to determine stability of a vaccine.

### EXAMPLES

### Example 1. General Protocol for Vaccine Sample Testing

In accordance with the methods and systems described herein, a vaccine sample or standard sample containing a target of interest that may be comprised of whole virus, split virus, recombinant protein or other possible vaccine samples known to one of ordinary skill in the art was mixed with a diluent prior to addition to an array of the current invention. In some embodiments, the diluent may include a detergent, such as Zwittergent 3-14 or Triton X-100, depending upon the nature of the vaccine sample or standard sample to be analyzed.

In some embodiments, a blocking buffer solution is added to the sample and diluent combination to arrive at a final total solution volume, and the total solution was then added to a pre-washed array. Substrates on which arrays containing samples to be analyzed were then incubated in a humidity chamber at room temperature for a target-specific time period (e.g. 1 hour). After incubation, excess solution was removed from the array(s) by pipette. A label agent solution appropriate for the target being investigated was added to the array(s). The array(s) were then further incubated in a saturated humidity environment for 30 minutes at room temperature. After the incubation, excess label agent solution was removed by pipette and the substrate was washed with an initial wash buffer, with excess wash buffer then removed. The substrate was then washed with a second wash buffer, with excess wash buffer again removed. The substrate was then washed with a 70% ethanol in water solution, with excess wash solution again removed, and finally washed with a purified water solution. Substrates were subsequently dried via forced air, pipette removal of remaining water, or passive drying.

Substrates were then imaged using a fluorescence microarray scanner and the quantitative data extracted for downstream analysis.

### Example 2: Antibody Capture Agent Screening Process and Anti-Measles Antibody Capture Agent Reactivity and Sensitivity

To arrive at an array in accordance with an embodiment of the current invention, a wide range of monoclonal antibodies were evaluated for specificity for use as capture agents. Antibodies were selected from a wide range of commercial sources for the ability to bind measles and rubella targets. Measles targets potentially targeted by the capture agents include nucleoprotein (NP), hemagglutinin (HA) and fusion protein (F). Rubella targets potentially targeted by the capture agents include E1 protein, E2 protein, and capsid (C) protein. In some cases, the target protein of the monoclonal antibody is not known, but rather was developed against a whole virus.

Antibodies were received and diluted using phosphate buffered saline (PBS), glycerol, CHAPS, and Nexterion P spotting buffer (Schott) at antibody concentrations ranging from 100 to 200 μg/mL depending on the antibody, and each antibody spotted onto appropriately functionalized glass substrates, such as epoxide functionalized glass. Triplicate spots of each antibody were printed in each array, as shown schematically in FIG. 1. After printing, the substrates were post-processed including a humidification step, an adhesive gasket added to create individual reaction wells for each replicate array, a stabilizing agent containing PBS and bovine serum albumin applied to protect the array and capture agents from degradation, and the glass substrates containing the replicate arrays packaged in the presence of an inert gas and a desicant to further prevent degradation.

To screen the anti-measles antibody capture agents for reactivity and sensitivity, an appropriate volume of gamma-irradiated measles antigen was diluted to 360 μg/mL of total protein, and a serial dilution of each antigen down to ˜20 μg/mL was created. The substrates containing the arrays were incubated in a humidity chamber at room temperature for two hours on an orbital shaking with agitation at approximately 65 rotations per minute. Excess material is removed by pipette and the substrates washed. An appropriate mixture of fluorophore-conjugated label agents was added to each array and incubated at room temperature. Excess label agent was removed and the substrate was washed. The processed arrays were then imaged on a fluorescence microarray scanner. Quantitative fluorescence data was extracted and processed. The data processing was automated with a commercially-available software package but can also be performed manually.

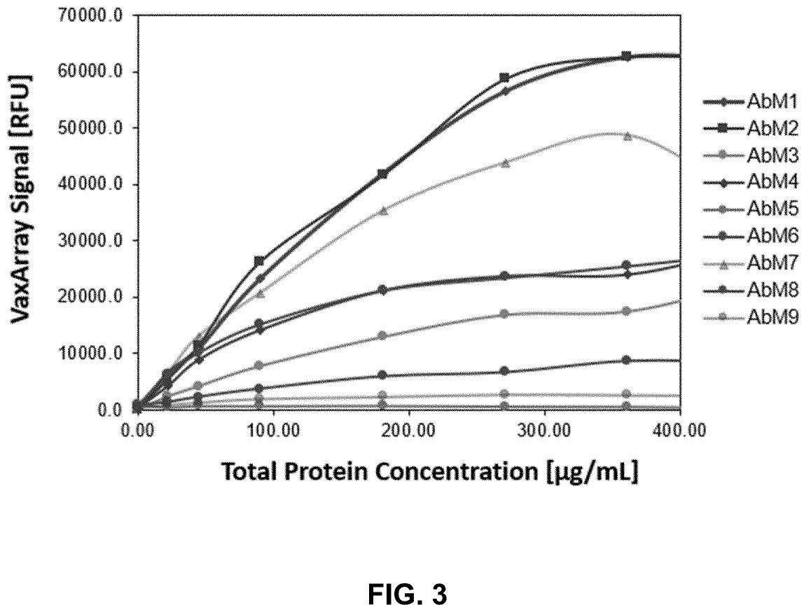

The dilution series for each antibody screened were then plotted as shown in FIG. 3. The nine measles antibody capture agents screened are labeled AbM1 through AbM9, and the data in FIG. 3 shows that the antibody capture agents screened resulted in a range of reactivities and sensitivities. AbM1 and AbM2 showed the highest sensitivity as demonstrated by the highest slope of the serial dilution. A similar process to that outlined above was utilized to screen the anti-Rubella antibody capture agents obtained.

As an alternative to the use of commercially-available antibodies as capture agents, one can develop custom antibodies as capture agents to arrive at an array in accordance with an embodiment of the current invention. Custom antibodies can be developed by the traditional hybridoma method involving immunization of mice to produce antibodies, isolation of antibody-producing cells, and fusion of antibody-producing cells with myeloma cells to produce hybridomas. Clones are then screened for the desired characteristics, and then scaled up and expanded. Alternatively, custom antibodies can be developed recombinantly. Alternatively, affirmers or aptamers can be developed using the appropriate corresponding technologies and utilized as capture agents.

### Example 3. Anti-Rubella Antibody Capture Agent Reactivity, Specificity and Rubella Target Quantification

To arrive at an array of the current invention, the reactivity of all anti-rubella antibody capture agents printed on the array was investigated. A rubella-containing sample was mixed with phosphate buffered saline (PBS) and a Zwittergent 3-14 detergent in water solution to create a final 1% Zwittergent (v/v) with antigen solution. The final rubella-containing sample was then processed via the general array processing protocol described in Example 1. As highlighted in FIG. 4, referencing the array layout in FIG. 1, and indicated by the intensity of the triplicate microarray spots (sub-array), several of the anti-rubella antibody capture agents produced significant signal on the array, demonstrating good reactivity. Specifically, anti-rubella antibodies AbR3, AbR4, and AbR10 (referring to FIG. 1) produced the highest fluorescence intensities. FIG. 4 exemplifies a pattern of capture agent-target complexes, where the pattern is an optical readout. Similarly, FIG. 6 illustrates another pattern of capture agent-target complexes, illustrating detection of targets that are different than the targets of the FIG. 4. If the sample contains both measles and rubella targets, the optical read-out is a combination of the patterns of FIGS. 4 and 6. Measuring the intensity of the spots, in combination with a calibration curve, provides quantification of targets, thereby providing valuable information about the sample, including for quality control, optimization and efficacy testing.

To evaluate the specificity of the anti-rubella antibody capture agents to rubella target, also known as an absence of cross-reactivity with measles target, a measles-containing sample was prepared for analysis to examine signal on the anti-rubella antibody capture agents. The measles-containing sample was combined with PBS and a Zwittergent 3-14 solution to generate a final 1% Zwittergent (v/v) with antigen solution. The final rubella-containing sample was then processed via the general array processing protocol described in Example 1.

Each anti-rubella antibody capture agent on the array layout shown schematically in FIG. 1 was evaluated for its reactivity with measles target. None of the anti-rubella antibody capture agents produced fluorescence intensity significantly above background signal when in the presence of a measles-containing sample. This indicated the anti-rubella antibody capture agents are not cross-reactive with measles target and are therefore specific for rubella.

To evaluate the anti-rubella antibody capture agents for the ability to quantify rubella target, a serial dilution of a rubella target was prepared and processed according to the general array processing protocol described in Example 1. Specifically, each dilution of the rubella target sample was added to one of the replicate arrays on a substrate according to the methods and systems of the current invention.

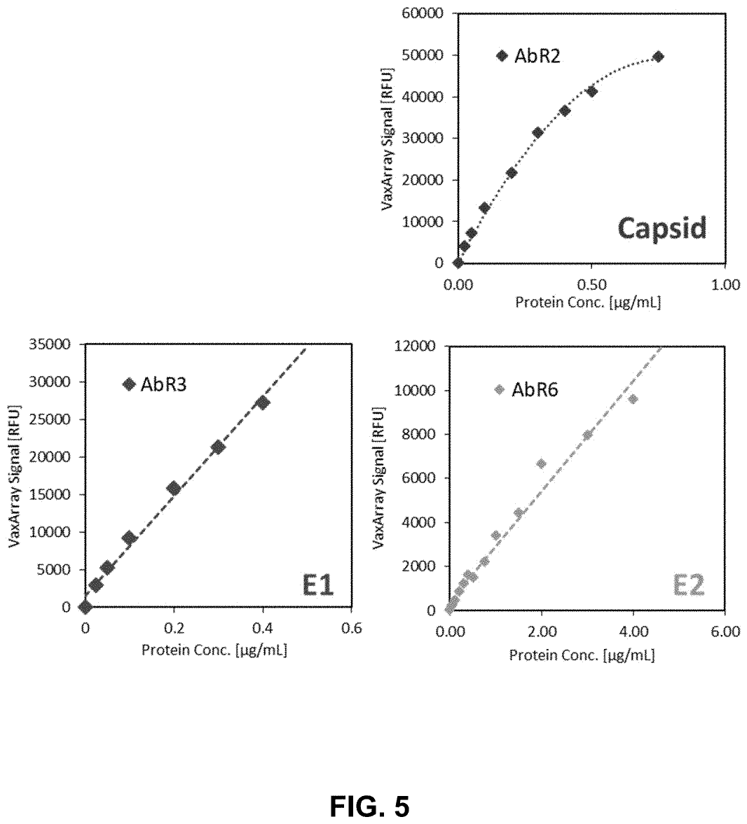

To prepare a response curve from the rubella antigen serial dilutions, the median signal from the 3 replicate spots for the anti-rubella antibody capture agent was calculated and plotted against the known antigen concentration in each array based on the known dilution factor applied to generate each sample. The dilution curves for this analysis are shown in FIG. 5. Assignment of protein target for the response curves of: rubella E1 protein (bottom left), E2 protein (bottom right), and capsid protein (top right) were based upon information provided by the antibody manufacturer or supplier.

The dilution curves for each printed antibody capture agent were evaluated for linearity by assessing the: i) R² coefficient of a best-fit line through the data series, and ii) dynamic range as defined as the span of concentrations at which the antibody is responding linearly to the change in target concentration and a sample of unknown rubella target concentration could be reliably quantified. Based on the data in FIG. 5, good linearity was obtained from AbR2, AbR3, and AbR6 (referencing the antibody labeling in FIG. 1), and the linear dynamic ranges for AbR2, AbR3, and AbR6 were 0.025-0.5 μg/mL, 0.025-0.4 μg/mL, and 0.5-4.0 μg/mL, respectively.

### Example 4: Anti-Measles Antibody Capture Agent Specificity and Measles Target

### Quantification

To arrive at an array consistent with the current invention, the specificity of the printed anti-measles antibody capture agents to measles target was investigated. A rubella-containing antigen sample was combined with PBS and a Zwittergent 3-14 solution to generate a final 1% Zwittergent (v/v) with antigen solution and analyzed by the system and methods consistent with the current invention via the general array processing protocol described in Example 1.

Each anti-measles antibody capture agent on the array layout shown schematically in FIG. 1 was evaluated for its reactivity with rubella target. None of the anti-measles antibody capture agents produced fluorescence intensity significantly above background signal when in the presence of a rubella-containing sample, as shown in the example image in FIG. 4, indicating that the anti-measles antibody capture agents are not cross-reactive with rubella target. The data in FIG. 6 alternatively show high fluorescence intensity of several of the anti-measles antibody capture agents when measles target is applied to the array, demonstrating reactivity for measles.

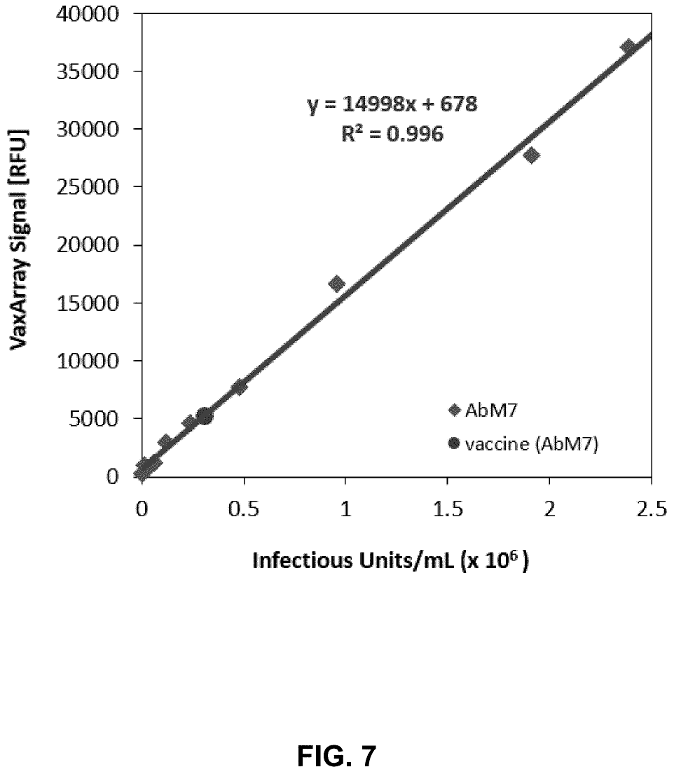

To prepare a response curve for the measles-containing serial dilutions, the median signal from the 3 replicate spots on the AbM7 anti-measles antibody capture agent (see FIG. 1 for numbering scheme) was calculated and plotted against the known antigen concentration in each array based on the known dilution factor applied to generate each sample. The dilution curve for this analysis is shown in FIG. 7.

The dilution curve was evaluated for linearity by assessing the i) R² coefficient of a best-fit line through the data series, and ii) dynamic range as defined as the span of concentrations at which the antibody is responding linearly to the change in antigen concentration and quantification of a sample of unknown measles antigen concentration could be reliably quantified.

Antigen-dilution curves for each printed antibody were evaluated for: i) linearity based upon the R² coefficient of a best-fit line through the data series and ii) dynamic range as defined as the span of concentrations at which the antibody is responding linearly to the change in antigen concentration and quantification of a sample of unknown measles target concentration could be reliably quantified. Based on the data in FIG. 7, good linearity was obtained for measles antigen using the AbM7 anti-measles antibody capture agent as demonstrated by a high R² coefficient of 0.996. The linear dynamic range was determined to be 0.25×10⁶ to >2.5×10⁶ infectious units/mL

### Example 5: Rubella Comparative Analysis

A rubella-containing sample with known tissue culture infectious dose (TCID50) titer was diluted to four different concentrations and blinded to the scientist executing each of the studies that follow. Each blinded dilution of the rubella-containing sample was tested using: 1) the system and methods of the current invention, 2) real-time reverse transcription polymerase chain reaction (rRT-PCR), and 3) TCID50 to compare performance.

In accordance with the methods and systems of the current invention, a rubella-containing sample was serially diluted in blocking buffer to generate seven total solutions to be utilized as a calibration curve. The four blinded rubella-containing samples were diluted further in blocking buffer. The serial dilutions, blinded rubella-containing samples (each analyzed in 6 replicate arrays), and a blank array (to which a solution of blocking buffer (no target present) was applied) were all processed in accordance with the current invention via the general array processing protocol described in Example 1. A calibration curve was then generated using the signal intestines of the seven serially diluted samples and the blank sample and plotting them against their known TCID50 titers. The signal intestines for each replicate of each blinded sample were then fit to the calibration curve to determine the unknown concentration of rubella target present in terms of equivalent TCID50 values expressed in infectious units per milliliter. The TCID50 equivalent titer values were averaged across the 6 replicates for each blinded sample, and the average was reported for each blinded sample. These values were then unblinded and compared to the expected concentrations based on the dilutions that were performed.

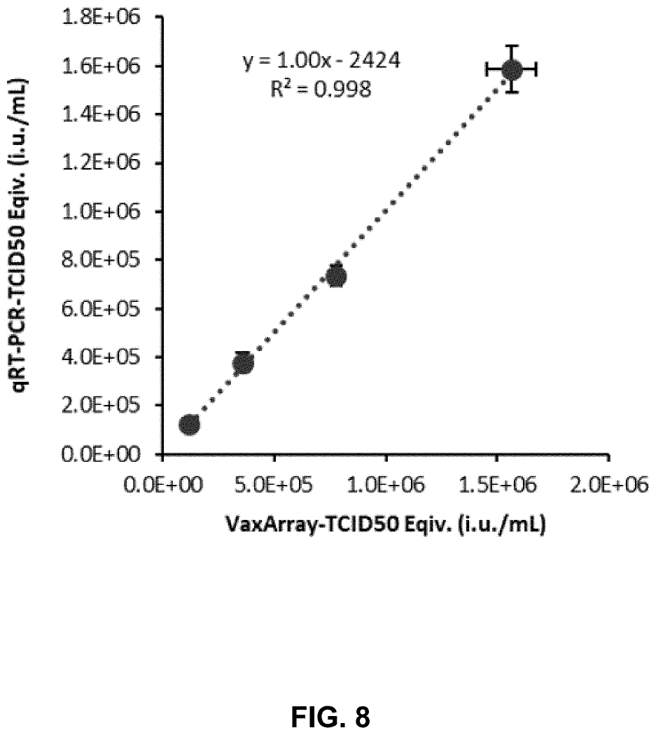

To conduct a rRT-PCR analysis on the blinded rubella-containing samples, a serial dilution of the rubella-containing sample of known titer and four blinded samples were each diluted 2-fold and the RNA from each was extracted using QlAamp MinElute Virus Spin Kit (Qiagen, 57704 and amplified using the SuperScript III Platinum One-Step qRT-PCR Kit w/ROX (lnvitrogen, #11745100). Following extraction, each extract was run in 3 replicates in a single rRT-PCR assay. Primers and probes designed to amplify a region of the rubella genome were obtained from the literature (Ammour, et al, Journal of Virological Methods, 2013, 187(1), 57-64.). Reverse transcription proceeded in a Stratagene MX3005p instrument at 50° C. for 30 min. followed by 95° C. for 2 min, followed by 45 cycles of PCR at 95° C. for 15 sec (melt). 58° C. for 33 sec (anneal/extend). A calibration curve was generated using the average Ct values of each serial dilution plotted against the known TCID50 for each dilution. The Ct values for each replicate of each blinded sample was then fit to the calibration curve to calculate rRT-PCR equivalent titer values in infectious units per milliliter. The rRT-PCR determined TCID50 equivalent titer values were averaged across the three replicates for each blinded sample and the average was reported for each blinded sample. The values were unblinded and compared to the expected concentrations based on the dilutions that were performed. In FIG. 8 is the correlation between the calculated rRT-PCR determined TCID50 equivalent and the TCID50 equivalents produced using the system and methods of the current invention. As can be seen from the very strong correlation coefficient (R2>0.99) and slope equal to 1, the rRT-PCR method is highly correlated to the array-based system and methods of the current invention.

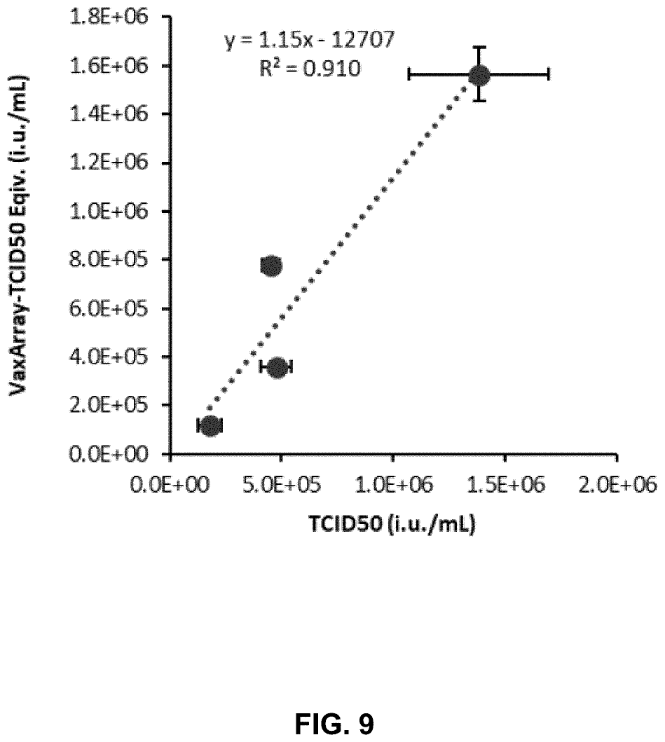

To conduct TCID50 analysis on the blinded rubella-containing samples, three separate 12-point serial dilutions were analyzed for each of the four blinded samples using a standard TCID50 protocol for monitoring cytopathic effect (CPE). The rubella samples were serial diluted using DMEM base medium supplemented with FBS, L-Glutamine, and Penicillin-Streptomycin and added to 96-well plates with RK13 cells. The plates were incubated in a CO₂ incubator at 32° C. Final readings of CPE formation were taken 10 days post-infection. Each replicate of each serial dilution for the 4 blinded samples was tested in 8 individual wells. After 10 days, CPE was observed and TCID50 titer in infectious units per milliliter was calculated for replicate using the Spearman-Karber method. The TCID50 titer values were averaged across the three replicates for each blinded sample and the average was reported for each blinded samples. The values were unblinded and compared to the expected concentrations based on the dilutions that were performed. In FIG. 9 is the correlation between the TCID50 assay results and the TCID50 equivalent values produced using the system and methods of the current invention. As can be seen from the reasonable correlation coefficient (R2=0.91) and slope close to 1, the rRT-PCR method is highly correlated to the array-based system and methods of the current invention. Given the known and expected irreproducibility/imprecision in the TCID50 method, it is not surprising that this correlation to the TCID50 assay is not as strong as that shown in FIG. 8 for the rRT-PCR analysis.

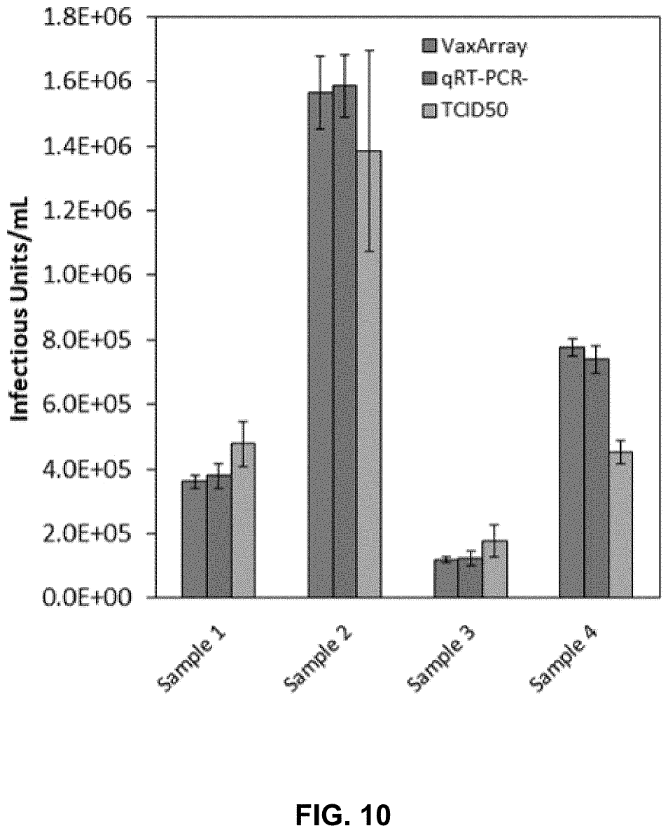

The determined titer in TCID50 equivalent infectious units/mL for the four blinded samples determined by the array-based system and methods of the current invention, rRT-PCR, and TCID50 were all quite similar is reported in FIG. 10.

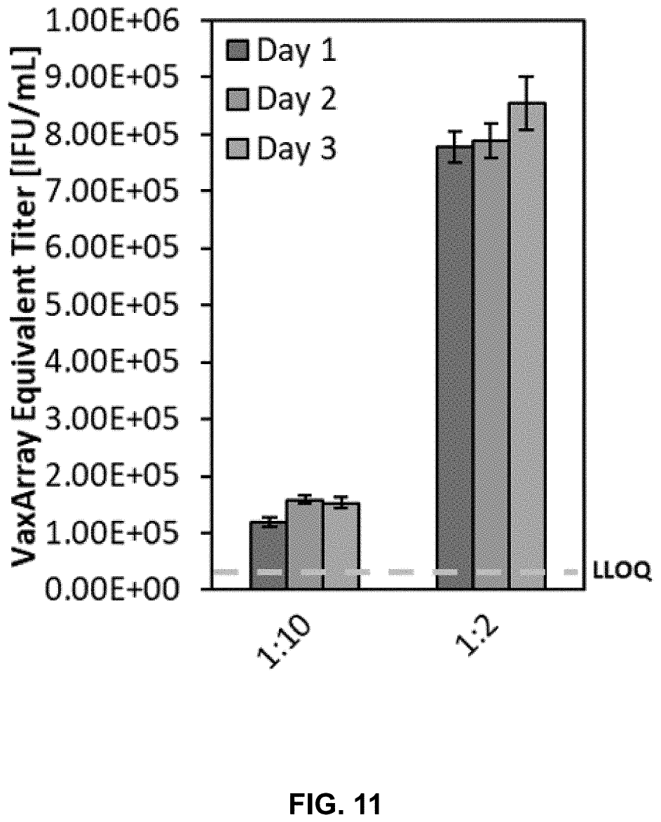

To assess the reproducibility of the system and methods of the current invention using a rubella-containing sample as an example, the system and methods of the current invention was repeated on three separate days using two blinded samples of the rubella-containing sample. Upon unblinding, it was determined these samples were a 1:2 and a 1:10 dilution of the sample. Both dilutions were assessed in 6 replicates. The average TCID50 equivalent values with associated error bars obtained using the system and methods of the current invention in infectious units per milliliter are presented for each dilution from each day in FIG. 11. Error bars are ±1 standard deviation of the replicates tested.

In a very similar set of comparative experiments, a measles-containing sample with known tissue culture infectious dose (TCID50) titer was diluted to four different concentrations and blinded to the scientist executing each of the studies that follow. Each blinded dilution of the measles-containing sample was tested using: 1) the system and methods of the current invention, 2) real-time reverse transcription polymerase chain reaction (rRT-PCR), and 3) TCID50 to compare performance.

The experiment involving the systems and methods of the current invention was conducted identically for the measles-containing samples as was just described for rubella. For rRT-PCR, the experimental setup and execution for the measles-containing samples was identical to that for rubella except that the primers and probe utilized in the rRT-PCR experiment were obtained specifically designed to amplify a region of the measles genome (Ammour, et al, Journal of Virological Methods, 2013, 187(1), 57-64). The TCID50 analysis was also identical to that conducted for rubella, except that the measles TCID50 analysis was conducted in Vero cell line, M199 base medium supplemented with FBS, L-Glutamine, and Penicillin-Streptomycin.

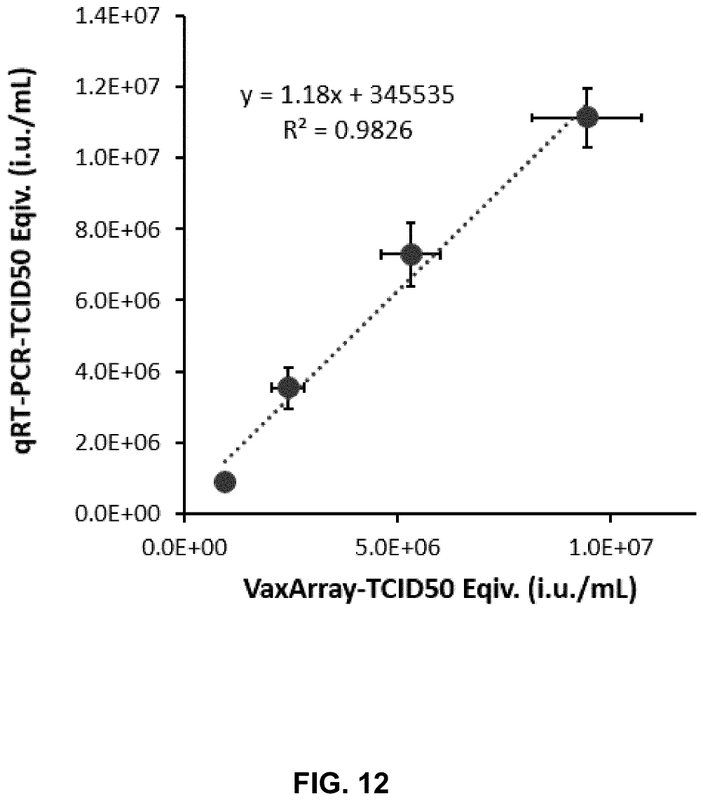

The relationship between the TCID50 equivalent titer values determined via the systems and methods of the current invention and the corresponding rRT-PCR values for the measles-containing samples are demonstrated in FIG. 12. High correlation was observed over the tested range, with slope of 1.18 and an r2 value of 0.9826.

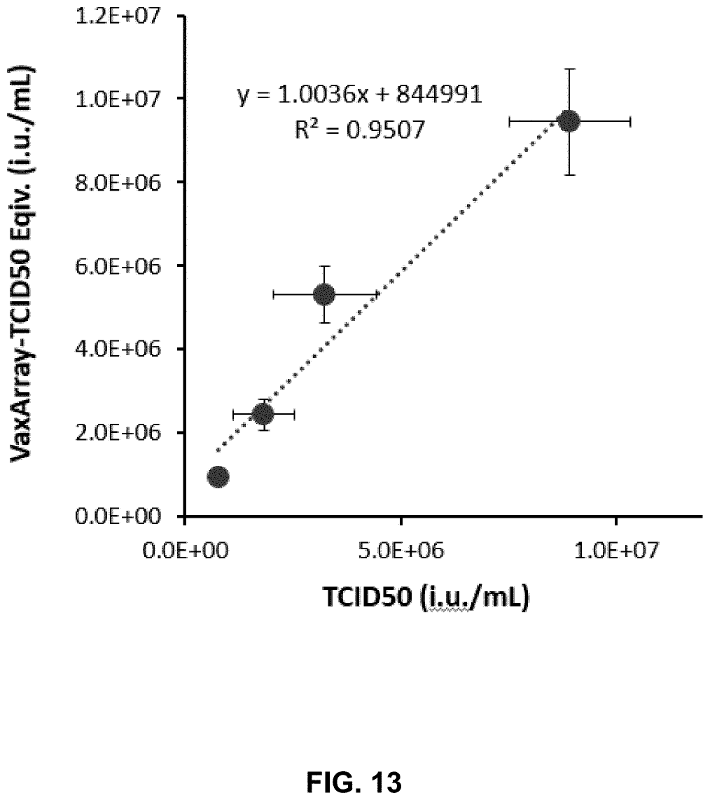

The relationship between TCID50 equivalent titer values determined via the systems and methods of the current invention and the values determined by TCID50 assay are shown in FIG. 13. A good correlation was observed over the tested range, with slope of 1.006 and an r2 value of 0.9507.

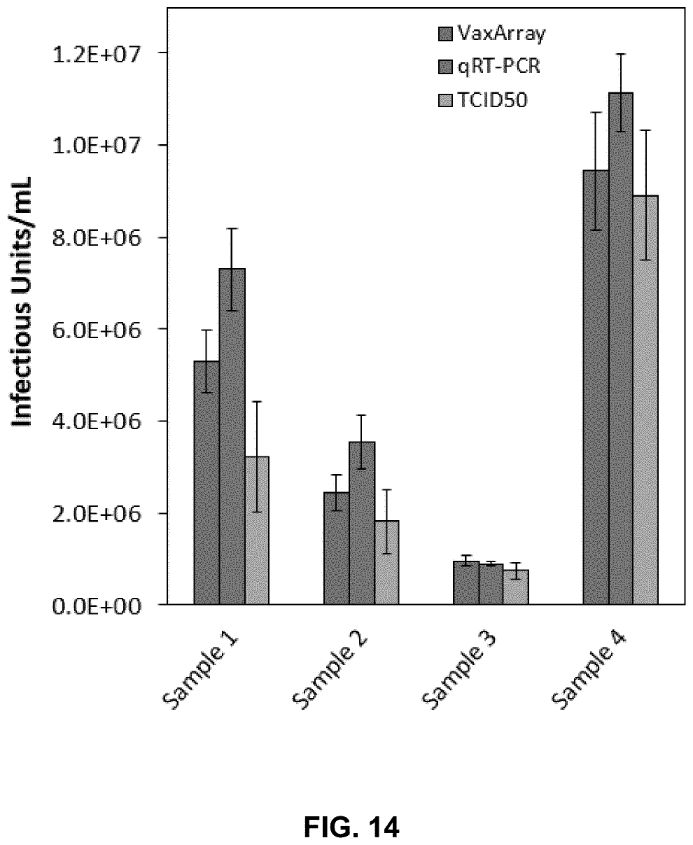

The determined titer in TCID50 equivalent infectious units/mL for the measles-containing samples determined by the array-based system and methods of the current invention, rRT-PCR, and TCID50 were all similar for the 4 blinded samples analyzed as reported in FIG. 14.

### Example 6: Simultaneous Quantification of Measles and Rubella

An important aspect of the systems and methods of the current invention is the ability to simultaneously quantify both measles and rubella in a single sample and to arrive at a similar result to that achieved when measles and rubella are run separately or monovalently. A bivalent mixture of both measles and rubella-containing samples was prepared alongside matched monovalent samples of rubella-containing and measles-containing samples. Serial dilutions of all 3 samples were prepared, and all were analyzed by the general array processing protocol described in Example 1.

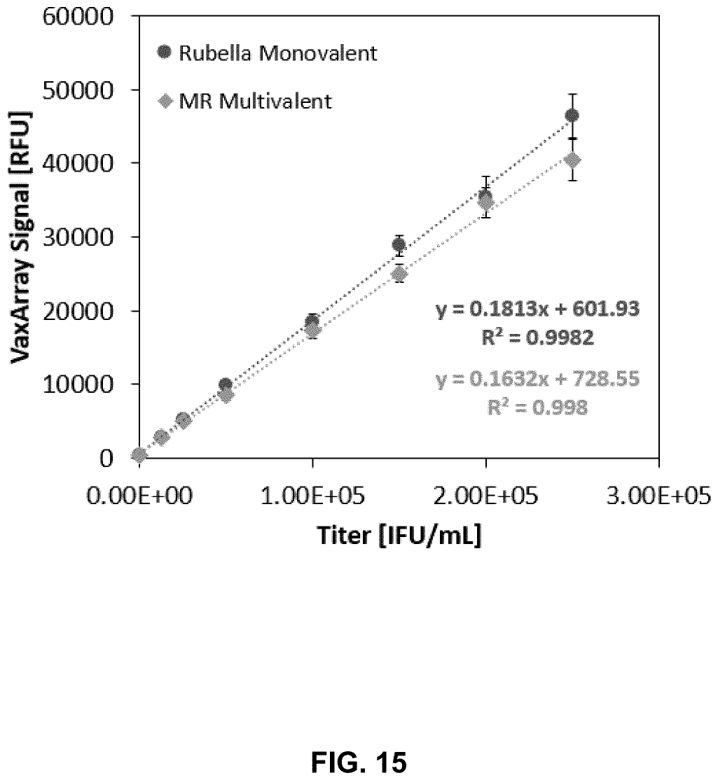

The fluorescence intensities for the rubella only dilution series detected using anti-rubella antibody capture agent AbR7 (referring to numbering in FIG. 1) were compared to the fluorescence intensities for the rubella plus measles bivalent dilution series for the same antibody capture agent. FIG. 15 shows the two serial dilutions and indicates that the rubella response curve is quite similar between the monovalent rubella serial dilution and the bivalent rubella plus measles serial dilution. These data indicate an ability to successfully quantify rubella target in the presence of measles target.

The fluorescence intensities for the measles only dilution series detected using anti-measles antibody capture agent AbM1 (referring to numbering in FIG. 1) were compared to the fluorescence intensities for the rubella plus measles bivalent dilution series for the same antibody capture agent. This testing indicated that the monovalent measles-containing sample resulted in a reduced fluorescence signal on the anti-measles antibody capture agent for the bivalent sample compared to the monovalent measles-containing sample. EXAMPLE 8 describes a methodology by which a measles-containing sample is pre-processed in accordance with systems and methods of the current invention to eliminate this interference.

A measles-containing sample was pre-processed using the methodology described in Example 7 to remove the substances that may be interfering with accurate quantification of measles target in the presence of rubella target. After pre-processing, the monovalent measles-containing sample was combined with a monovalent rubella-containing sample that had not been subject to the pre-processing described in Example 8. Serial dilutions were then made for both the monovalent and bivalent samples, and all samples were subjected to the general array processing protocol described in Example 1. Note that the concentrations of each component in the bivalent sample was matched prior to creating serial dilutions to enable a direct comparison between the dilutions.

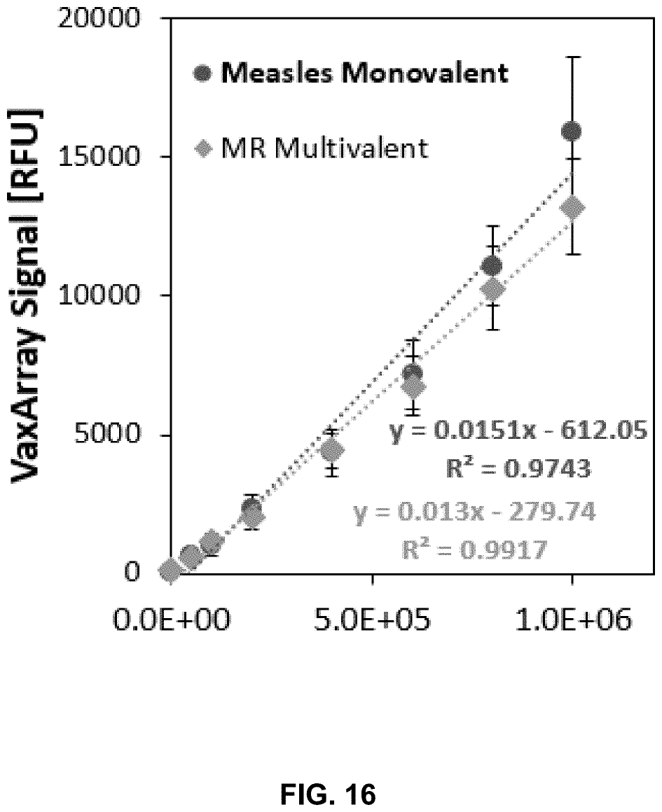

The median fluorescent signal intensity for the monovalent and bivalent serial dilutions on anti-measles antibody capture agent AbM1 (referring to numbering in FIG. 1) were then plotted against the known measles target concentration in each dilution. FIG. 16 compares the serial dilution for the measles component in the monovalent and bivalent samples and indicates that the measles target response curve is quite similar between the monovalent measles serial dilution and the bivalent rubella plus measles serial dilution. While the measles target was not accurately quantified using anti-measles antibody capture agent AbM1 (referring to numbering in FIG. 1) in the absence of a pre-processing step, the addition of a pre-processing step as outlined in Example 7 indicated that the pre-processing step allowed similar measles target response curves to be generated for both the monovalent and bivalent samples.

### Example 7: Pre-Processing

In accordance with the systems and methods of the current invention, a pre-concentration and cleanup step was developed. Optimization of methods using magnetic microbeads that bind the virus or using non-magnetic microbeads that bind the virus (product name obtained from were undertaken, with variables optimized including lysis solution and lysis time, ratio of microbeads to initial sample volume and concentration, incubation times, and separation conditions such as centrifugation speed.

An optimized procedure using the non-magnetic microbeads to provide high recovery was identified, and is as follows: i) microbeads in storage buffer were prepared for use by centrifuging at 21,000×g for 5 minutes, ii) the storage buffer supernatant was removed by pipette, iii) microbeads were resuspended in a measles-containing sample, iv) sample was incubated at room temperature for 30 minutes with occasional manual mixing by inverting the vial, v) centrifuged at 21,000×g for 5 minutes to pellet the microbeads, vi) the supernatant was removed by pipette, vii) microbeads were resuspended with a lysis solution containing 3% (w/v) Zwittergent 3-14 in PBS, viii) microbeads were incubated with lysis solution for 15 minutes with occasional manual mixing, ix) the solution was centrifuged at 21,000×g for 7 minutes to pellet the microbeads, x) and supernatant was then removed and subjected to downstream analysis or stored for future use.

An optimized procedure using magnetic microbeads was identified, and is as follows: i) microbeads in storage buffer were separated from the supernatant by placing the vial in a magnetic field for 1 minute ii) storage buffer was removed by pipette, iii) microbeads were resuspended with a measles-containing sample, iv) sample was incubated at room temperature for 30 minutes with occasional manual mixing by inverting the vial, v) microbeads were isolated by placing the vial in a magnetic field for 1 minute, vi) supernatant was removed by pipette, vii) microbeads were resuspended in a lysis solution containing 3% w/v Zwittergent 3-14 in PBS, viii) microbeads were incubated with lysis solution for 15 minutes with occasional manual mixing, ix) microbeads were isolated by placing the vial in a magnetic field for 1 minute, x) and supernatant was then removed and subjected to downstream analysis or stored for future use.

In the assessment of the optimized pre-processing protocols described above, four separate preparations of each microbead type (magnetic and non-magnetic) were evaluated via the respective optimized methods described above. Specifically, 80 μL of microbead solution was added to each tube. Beads were isolated as described above and the supernatant removed. A single preparation of a measles-containing sample was prepared in PBS, with a separate vial of 100 μL of the prepared measles-containing sample was stored separately as a control sample and not subjected to the pre-concentration and cleanup step. To each vial containing isolated microbeads, 800 μL of prepared measles-containing sample was added and the protocols described above followed for the magnetic and non-magnetic microbeads.

After isolation of the concentrated supernatant, the pre-processed samples and the non-treated control sample were subjected to the general array processing procedure outlined in Example 1, with each of the four separate pre-processed preparations and the non-treated control analyzed in triplicate. A measles-containing sample was utilized to generate a serial dilution and also subjected to the general array processing procedure outlined in Example 1, with each serial dilution analyzed on a single array.

The fluorescence signal intensity for each antibody was obtained for all samples. A calibration curve was constructed using the serial dilution data, and the concentration of measles target in each of the pre-processed samples was back calculated using the equation of the calibration curve.

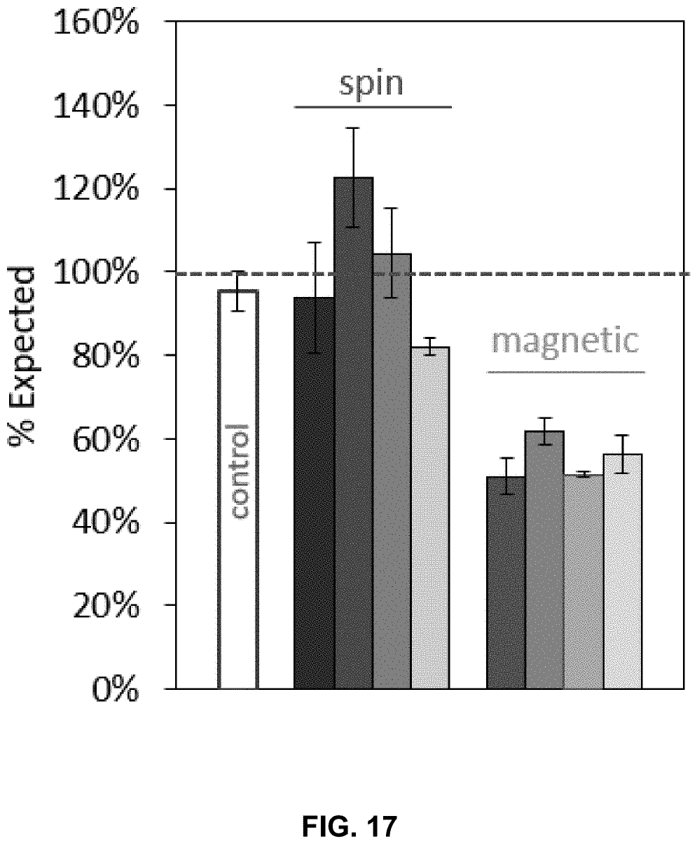

The dilution factors applied to the samples were accounted for, and the percent of the expected value (% Expected), often referred to as % recovery, was determined for each sample based on the value of the control. The average % Expected (% recovery) was determined for each procedure (magnetic and non-magnetic beads/“spin”) by averaging the 3 replicate measurements. Results of this analysis for the spin-based non-magnetic bead procedure and the magnetic bead procedure are shown in FIG. 17. Error bars represent ±1 standard deviation of the 3 replicate measurements. The data in FIG. 17 show that the spin-based method provides high recovery but less repeatability, whereas the magnetic bead-based procedure shows lower recovery but is more repeatable.

### Example 8: Stability Indication

To arrive at the systems and methods in accordance with the current invention in which stability indicating behavior of the antibody capture agents is desired, anti-rubella and anti-measles antibody capture agents consistent with the current invention were investigated track with the stability of measles and rubella targets. Using methods common to the vaccine industry and generally known in the art, a measles-containing sample and a rubella-containing sample were prepared in glass vials and subjected to a degradation protocol by placing both vials in a water bath set to 55° C. to degrade the proteins comprising the samples. After 4 hours, the glass vials were removed from the water bath.

The degraded samples along with controls of the measles and rubella samples not subjected to the degradation protocol were subjected to the general array-based protocol described in Example 1. Specifically, the measles-containing samples were combined with PBS and a Zwittergent 3-14 solution to a final 1% w/v Zwittergent content. The rubella-containing samples were combined with PBS and an IGEPAL CA-630 solution to a final 5% w/v IGEPAL content. Each sample was further diluted in blocking buffer plus detergent made to match the detergent contents of the lysed samples prior to executing the remainder of the array-based procedure.

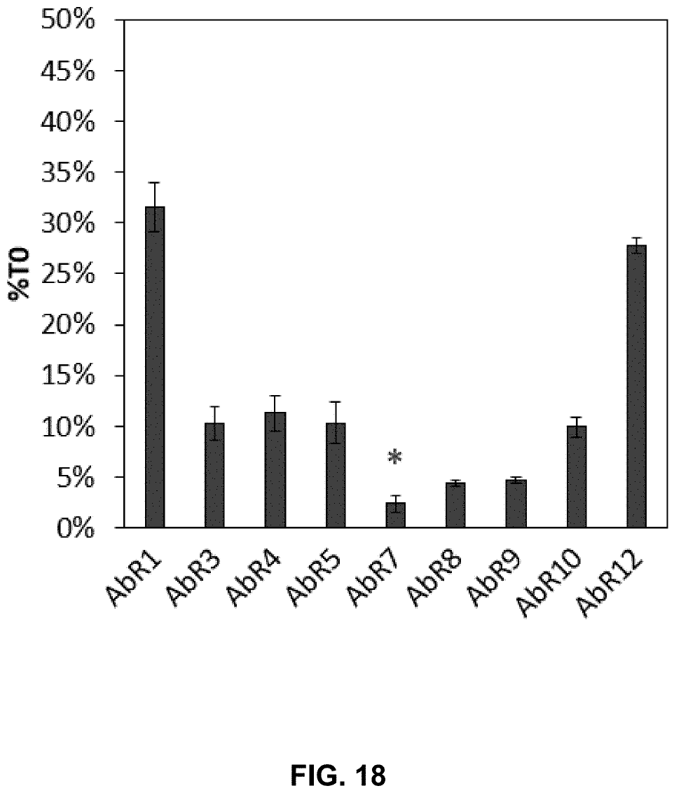

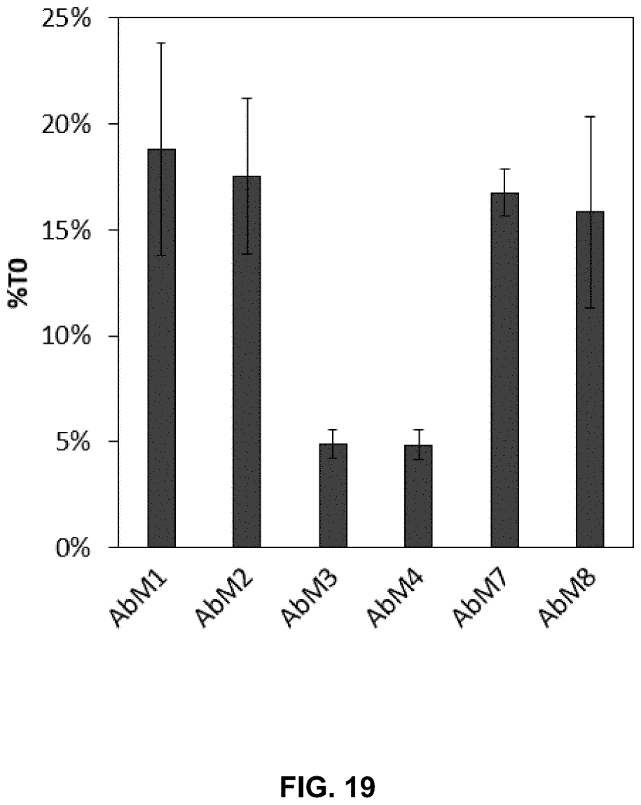

The average signal intensity on each antibody that resulted in signal intensities that were at least 3× background for the non-degraded control. for each dilution was then plotted as the percent change in signal due to degradation of target, or % T0. % T0 is calculated as the percentage of signal resulting from a degraded sample as compared to a matched control sample not subject to degradation, with the results for the anti-rubella antibody capture agents shown in FIG. 18 and the results for the anti-measles antibody capture agents shown in FIG. 19. Error bars shown represent ±1 standard deviation of the triplicate measurements, propagated.

As indicated in FIG. 18, all anti-rubella antibody capture agents shown demonstrated a decrease in signal of between 68% and 98% after the rubella-containing sample was degraded. As indicated in FIG. 19, all anti-measles antibody capture agents shown demonstrated a decrease in signal of between 81% and 95% after the measles-containing sample was degraded, indicating appropriateness of the antibody capture agents shown for detecting target stability.

### Example 9: Pre-Processing Via Virus Growth

The current gold standard method for qualifying and quantifying antigen content in measles and rubella vaccines is measuring the infectious dose of each virus in the sample to be analyzed. It is desired that the technology of the current invention report measurements accurate relative to the infectious measurement of the sample. Provided herein are antibody capture agents to detect protein from measles and/or rubella viruses, whereas viral infectivity is dependent upon several additional factors such as membrane integrity and genome integrity. Therefore, it is feasible for a virus sample to contain intact protein but not contain infectious virus particles or to have a different ratio of total protein to infectious virus particles. It is therefore a desired property to be able to provide an accurate measurement compared to the gold standard infectious dose measurement for certain applications.

To ensure accurate measurement of infectivity, an optimized procedure for sample pre-treatment that removes non-infectious viral particles from the sample was developed. The optimized pre-processing procedure developed is as follows: first, i) mammalian cell cultures (Vero or RK13 cells for measles or rubella sample analysis, respectively) are prepared according to industry standard methods, ii) cells are plated in 96-well cell culture plates and are incubated overnight (20-24 hours) at 37° C. under 5% CO₂ to generate a cell monolayer in each well, iii) virus-containing samples for analysis are then prepared in phosphate buffered saline, mixed 1:1 with cell-culture diluent media, and samples are then added to separate wells of the prepared cell culture plates. Next, iv) samples are incubated on the cells for a pre-determined period of time, such as at 32° C. for rubella samples or 36° C. for measles samples under 5% CO₂. v) Sample supernatant is removed, the wells are washed with PBS, and diluent media is added to each well. vi) Plates are incubated at 32° C. for rubella samples or 36° C. for measles samples under 5% CO₂ for a pre-determined time. vii) Supernatant from each well is collected, each well is then washed with PBS, and the PBS is collected. Finally, viii) cells are removed from each well of the plate using Trypsin and are collected for downstream analysis by the technology of the current invention.

To demonstrate that this pre-process results in measurements that are accurate compared to the gold standard infectivity or infectious dose measurement, for each virus (measles or rubella), monovalent bulk samples containing infectious virus were each split into two aliquots. One aliquot of each sample was subjected to 37° C. for 24 hours to cause complete loss of infectivity, as it was previously demonstrated that this treatment rendered viral samples completely free of infectious particles but retained detectable protein. Next, five (5) unique samples were prepared by combining the heat degraded sample with intact (non-degraded) sample at ratios of degraded:non-degraded of 100:0, 75:25, 50:50; 25:75, and 0:100. A portion of each of the 5 samples was process by the pre-treatment process as described immediately above in Example 8, and the other portion of each of the 5 samples was kept at 4° C. for future analysis.

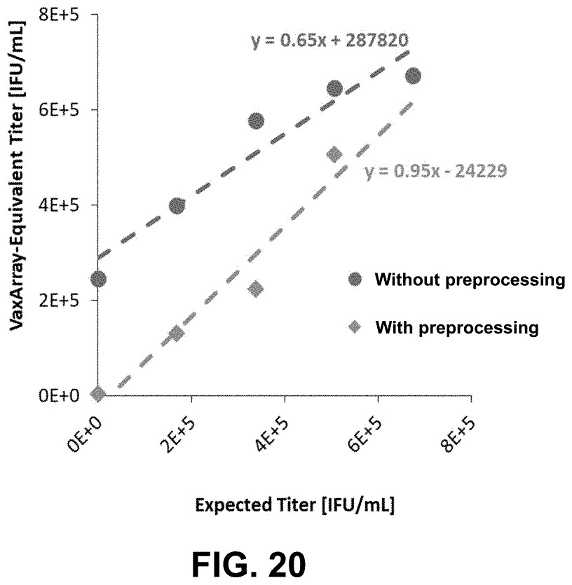

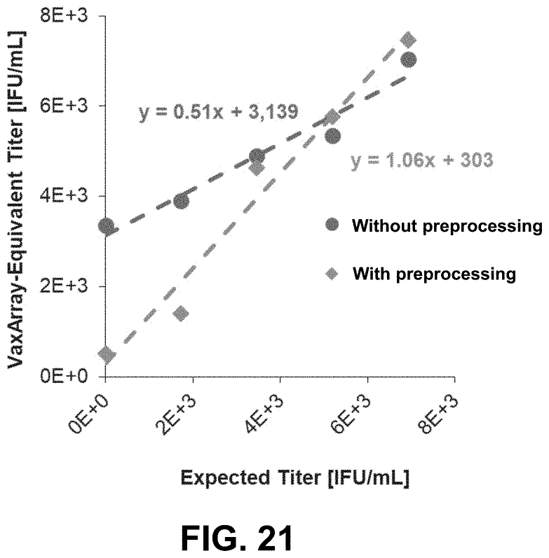

All 10 samples (5 that had been pre-processed by the method of Example 8, and 5 that were left untreated) were analyzed using the technology as described in Example 1. For each antibody capture agent that resulted in signal intensities that were at least 3× background for the 100% non-degraded sample, the average signal intensities obtained were plotted against the expected infectious titer of the sample measured by TCID₅₀. The data generated for this series of samples are shown in FIG. 20 and FIG. 21 for measles and rubella, respectively. Accurate is defined as measurements using the technology of the current invention that are equivalent to the expected value based on infectious dose. In FIG. 20 and FIG. 21, this equivalency would be shown as a regression line with a slope of 1 and a y-intercept of 0. Samples that were not subjected to the pre-processing method of Example 8 showed slopes of regression lines of only 0.51 for rubella (see FIG. 21, circles) and 0.65 for measles (see FIG. 20, circles), and the y-intercepts were 3139 and 287820, respectively. The samples that were pre-processed by the method of Example 8 prior to analysis by the invention resulted in slopes of regression lines of 1.06 for rubella (see FIG. 21, diamonds) and 0.95 for measles (see FIG. 20, diamonds), with associated y-intercepts of 303 and −24229, respectively. These data in FIG. 20 and FIG. 21 indicate that pre-processing of the samples in the manner described in Example 8 results in measurements that are accurate relative to the infectious dose.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Every device, system, combination of components, or method described or exemplified herein can be used to practice the invention, unless otherwise stated.

Whenever a range is given in the specification, for example, a physical dimension or a time range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

We claim:

1. A method for multiplexed detection of a plurality of targets associated with a measles virus and a rubella virus, the method comprising the steps of:
providing a plurality of capture agents specific to a measles target and a rubella target, wherein the capture agents specifically bind to the measles target and the rubella target;
contacting the plurality of capture agents with a sample, wherein measles target and rubella target in the sample form capture agent-target complexes; and
detecting a spatial pattern of capture agent-target complexes.

2. The method of claim 1, wherein:
the measles target is one or more of:
measles nucleoprotein (NP);
measles hemagglutinin (HA); and/or
measles fusion protein (F);
the rubella target is one or more of:
rubella E1 protein;
rubella E2 protein; and/or
rubella capsid protein (C).

3. The method of claim 1, wherein:
the measles target comprises NP; and
the rubella target comprises E1, E2 and C.

4. The method of claim 1, wherein the detecting step comprises providing an optical label agent that specifically binds to the capture agent-target complex.

5. The method of claim 1, wherein the capture agents comprise monoclonal antibodies.

6. The method of claim 5, wherein the monoclonal antibodies are selected to provide simultaneous detection of measles and rubella targets.

7. The method of claim 1, further comprising the step of quantifying multiple targets from a measles virus and/or a rubella virus.

8. The method of claim 1, further comprising the step of quantifying the multiple targets, wherein the sample is selected from the group consisting of: a measles vaccine; a rubella vaccine; a measles and rubella vaccine; an intermediate precursor of a measles and/or rubella vaccine obtained in a step of production of the measles and/or rubella vaccine.

9. The method of claim 1, wherein the target corresponds to an antigen, a protein or a fragment thereof, a virus, a virion, a virus-like particle, or a vaccine component.

10. The method of claim 1, wherein the sample is obtained during vaccine development or vaccine manufacture.

11. The method of claim 1, wherein a plurality of measles targets and a plurality of rubella targets are simultaneously detected and quantified.

12. The method of claim 1, wherein the capture agents are provided as a microarray in a plurality of wells in a multiple-well substrate; or a bead surface.

13. The method of any of claim 12, wherein the substrate comprises:
replicate microarrays;
individual microarray spots ranging from between 50 μm and 400 μm in diameter; and/or
each microarray spot contains a plurality number of copies of a single capture agent.

14. The method of claim 13, wherein the capture agents on the substrate form a microarray, and each capture agent is provided as a plurality of replicates, wherein each replicate corresponds to the spot.

15. The method of claim 1, further comprising the step of identifying and/or quantifying the measles targets and the rubella targets bound to the capture agents.

16. The method of claim 1, further comprising the step of applying a physical challenge to the sample and subsequently quantifying targets bound to the capture agents to identify degradation for each of the targets bound to the capture agents, wherein the one or more physical challenge is selected from the group consisting of: a temperature change, a pH change, a contaminant introduction, a chemical introduction, and a storage time period.

17. The method of claim 16, wherein the quantifying step comprises:
applying various known concentrations of a standardized target to corresponding replicates;
fluorescently labeling bound standardized target-capture agent complexes;
measuring a fluorescent signal from the fluorescently labeled complexes as a function of the standardized target concentration to generate a calibration curve; and
calculating a concentration of the targets from a fluorescence output associated with the corresponding capture agent-target complex.

18. The method of claim 17, used to determine one or more target concentrations during a vaccine manufacture process or vaccine optimization process.

19. The method of claim 1, further comprising the step of concentrating targets in the sample before the contacting step, wherein the concentrating step comprises:
mixing the sample with a cleanup matrix, wherein the target within the sample binds to the cleanup matrix;
applying a centrifugal and/or magnetic force to separate the sample bound to the cleanup matrix to form a supernatant over a concentrated sample bound to the cleanup matrix;
removing at least a portion of the supernatant;
resuspending the sample bound to the cleanup matrix in a lysis solution to remove target from the cleanup matrix; and
removing the cleanup matrix from the solution to thereby obtain a concentrated solution of targets.
